(19) United States
(12) Patent Application Publication
(10) Pub. No.: US 2005/0025789 A1
(43) Pub. Date: Feb. 3, 2005

(54) Allogenic vaccine that contains a costimulatory polypeptide-expresing tumor cell

(76) Inventors: John Nieland, Stockdorf (DE); Claudia Breidenstein, Neu-Esting (DE); Ute Sartorius, Berlin (DE); Ulrich Moebius, Gauting-Unterbrunn (DE); Christoph Bogedain, Munchen (DE); Adelheid Dinkel, Kufstein (AT)

Correspondence Address: CLARK & ELBING LLP, 101 FEDERAL STREET, BOSTON, MA, 02110, US

(21) Appl. No.: 10/494,715

(22) PCT Filed: Nov. 8, 2002
(86) PCT No.: PCT/EP02/12526

Related U.S. Application Data
(60) Provisional application No. 60/332,497, filed on Nov. 9, 2001.

(51) Int. Cl. A61K 39/00

(52) U.S. Cl. 424/277.1; 514/12

(57) ABSTRACT

The invention relates to the use of a tumor cell for producing a vaccine for the treatment or prophylaxis of tumors in patients, said tumor cell expressing a costimulatory polypeptide and said tumor cell and said patient having non-identical MHC molecules. The invention further relates to the use of a costimulatory polypeptide-expressing tumor cell for producing a vaccine for increasing the lytic activity of NK cells in the treatment or prophylaxis of a tumor in a patient that is allogenic with respect to the tumor cell.

The present invention relates to the use of genetically modified tumor cells for producing vaccines.

Activating the endogenous immune system for the purpose of treating and preventing tumors is a promising approach in modern cancer therapy.

The prior art discloses, inter alia, autologous and allogenic vaccines for the purpose of activating the endogenous immune system (Pardoll D. M., (1998) Nat.

Med. 4 (5 Suppl): 525-31; Wolchock J. D. and Livingston P. O., (2001) Lancet Ocol. 2 (4): 205-11; Schadendorf D. et al., (2000) Immunol. Lett. 15; 74 (1): 67-74).

In the case of autologous vaccines, cells from the patient's own tumor are used for producing the vaccine. In this connection, the tumor cells are removed from the body, genetically modified, where appropriate, and made proliferation-incompetent, for example by irradiation, before they are administered to the patient once again. The aim is for immune cells, in particular cytotoxic T cells and helper T cells, to recognize the cells which have been administered and, in this way, to build an immune response which can then also be directed against the tumor.

In addition to many advantages, autologous cell vaccines also possess a number of major disadvantages. Particularly in the case of relatively small neoplasias, it is frequently very difficult, or virtually impossible, to culture the tumor cells. In addition, it is necessary to prepare a vaccine individually for each patient. It is consequently very difficult to standardize the production of autologous vaccines, a situation which can represent a substantial disadvantage for the approval of such a vaccine. Furthermore, producing an autologous vaccine implies a long waiting time for the patient since, after the tumor material has been removed, the cells have first of all to be prepared and manipulated before they can be administered to the patient once again. In the meantime, the danger exists that (additional) metastases will have been formed in the patient's body.

An alternative to autologous vaccination is what is termed allogenic immunization, i.e. immunizing with cells which are not derived from the same patient. Consequently, the vaccine cells differ from the endogenous cells of the patient since they as a rule do not possess the identical transplantation antigens (MHC genes).

The MHC complex on the surface of cells is of particular importance for developing the specific immune response since peptides are presented in the MHC complex, with these peptides then being recognized by T cells which are specific for them. In this regard, there are two classes of MHC complexes, i.e. class I and class II.

When a specific immune response is developed, a T cell recognizes, by way of its T cell receptor, the MHC complex together with the presented peptide of an antigen and is thereby stimulated to develop an immune response. However, binding of the T cell receptor to the MHC complex is not usually sufficient for developing a specific immune response. Additional so-called costimulatory molecules are required, with these molecules amplifying the signal exchange between the T cell and the MHC-bearing cell.

The class I-MHC complexes are of particular importance for inducing an immune response against tumor cells since the latter present, in their MHC-I complexes, peptides which are found (almost) exclusively on tumor cells, i.e. what are termed tumor antigens, or peptides which are derived from these antigens. It is known in the prior art that the recognition, by particular T cells, of peptides which are derived from tumor antigens and which are presented by MHC class I molecules brings about the proliferation of cytotoxic T lymphocytes (also termed cytotoxic T cells) which are in turn able to destroy tumor cells (Janeway C. et al., (1999) in: Immunobiology; Current Biology Publications, 551-554).

In humans, there are three genes which encode three different MHC class I molecules, i.e. HLA A, HLA B and HLA C. Each of these genes is highly polymorphic, i.e. a number of different alleles, which lead to different MHC molecules, exist in the population in the case of each of the genes. For example, according to the present state of knowledge, there are 95 different HLA A, 207 HLA B and 50 HLA C alleles in the Caucasian population. While some of the alleles occur very frequently, for example the allele HLA A2, which occurs in approx. 50% of the population, other alleles occur only very rarely.

The HLA type of an individual can be determined by two different methods. In the first place, it is possible to obtain antibodies which specifically recognize particular MHC proteins, which are encoded by HLA alleles, and which are consequently used for specifically staining the cells of a test subject. In the second place, there are specific oligonucleotide primers for the different alleles, with these primers being used in PCR reactions for the purpose of determining the HLA type of a test subject. (Welsh K and Bunce. M (1999) Rev Immunogenet 1(2): 157-76; Parham P (1992) Eur J Immunogenet 19(5): 347-59).

If different, unrelated individuals are now compared, it is found that they frequently have no allele of the MHC I complex in common, such that it is said that there is no congruence between their MHC or HLA molecules. While the two individuals are allogenic when they have at least one HLA allele in common, their MHC or HLA molecules are partially congruent. Their MHC or HLA molecules are completely congruent when the two individuals possess all the HLA alleles in common, something which as a rule only occurs between close relations, in particular enzygotic twins.

In the state of the art, it is assumed that a particular T cell only recognizes one type of MHC complex, as a rule the endogenous MHC complex. This is due to the fact that, within the context of positively selecting T cells in thymus, i.e. the site of production of the T cells, the only T cells to survive are those which recognize endogenous MHC complexes. However, alloreactive T cells, which recognize foreign MHC complexes, for example on the cells of transplanted organs, are also present in the body.

This, and other, information can be gathered from the textbook “Immunobiology”, Charles Janeway et al., Current Biology Publications, 1999, pages 115-147, in particular pages 115-117 and 135-140.

In the case of allogenic vaccination, an (or else more than one) established tumor cell line(s) is/are as a rule used for vaccinating the patient (see WO 97/24132).

Although some degree of immune reaction is elicited in the patient's body simply by administering an allogenic tumor cell line, this immune reaction is as a rule insufficient for controlling the patient's own tumor.

For this reason, a variety of attempts have been made in the prior art to elicit an amplification of the immune response by genetically manipulating the tumor cell line which is administered. For example, the prior art (see WO 97/24132) discloses that an amplification of the immune response can be achieved by administering a genetically modified tumor cell which expresses GM-CSF. All in all, the prior art discloses a large number of allogenic vaccines which comprise genetically modified tumor cells. (Nawrocki S. et al. (2001) Expert Opin Biol Ther 1(2): 193-204.

Despite this large number of potential allogenic vaccines, there is no known allogenic vaccine in the prior art which achieves a satisfactory effect when used in a patient. A feature possessed in common by all the allogenic vaccines known in the prior art is that the immune response which is induced in the patient is as a rule too weak to effectively combat the patient's tumor (Bodey B. et al. (2000) Anticancer Res 20(4): 2665-76).

Consequently, the object of the present invention is to provide an improved allogenic vaccine which induces a more powerful immune response in the-patient than do the allogenic vaccines which are known in the prior art.

According to the invention, this object is achieved by using a tumor cell to produce a vaccine for treating or preventing a tumor in a patient, characterized in that the tumor cell expresses a costimulatory polypeptide and in that the tumor cell and the patient do not exhibit any congruence in their MHC molecules.

Surprisingly, it has been found, within the course of the present invention, that the activity of a vaccine without any congruence in the MHC complexes can be increased by the tumor cell expressing a costimulatory polypeptide. This leads to an increase in efficiency of approx. 30%, preferably of approx. 40%, a figure which, for example in the case of the very aggressive K1735 tumor, represents a very marked increase in efficiency.

This is so surprising because it has thus far been assumed in the prior art that costimulatory molecules can only bring about an increase in the immune response when the tumor cells, which are used as the allogenic vaccine, and the patient, and consequently the tumor as well, exhibit at least partial congruence between their MHC molecules. It has been assumed that the existence of a congruence in the MHC I complex is a prerequisite for allogenic tumor cell-activated T cells to be able to combat the endogenous MHC molecule-equipped tumor since, according to conventional opinion, the individual T cells are only able- to recognize one MHC type (Fabre J W (2001) Nature Medicine 7(6): 649-52.

As already explained above, the term “allogenic” means, within the context of the present invention, that two individuals (or one individual and the cell which is used for the vaccination) differ in regard to their antigens. As a rule, but not necessarily, this means that they differ in regard to their HLA antigens. In this connection, the possibility of the two individuals being partially congruent in their HLA genes is expressly included. Complete congruence, or no congruence, in the HLA genes is also included. In the former case, at least one further antigen then differs between the individuals (or the cell and the-patient).

The expression “no congruence between their MHC/HLA molecules” means that two individuals (or an individual and the cell used for the vaccination) have no alleles of their MHC I complexes in common.

According to a preferred embodiment, the costimulatory polypeptide is selected from the group comprising B7.1, B7.2, CD40, Light, Ox40, 4.1.BB, Icos L, SLAM, ICAM 1, LFA-3, B7.3, CD70, HSA, CD84, CD7, B7 RP-1 L, MAdCAM-1, VCAM-1, CS-1, CD82, CD30, CD120a, CD120b and TNFR-RP, CD40L.

According to a particularly preferred embodiment, the costimulatory polypeptide is selected from the group comprising B7.1 and B7.2. According to a very particularly preferred embodiment, the costimulatory polypeptide is B7.2.

According to a preferred embodiment of the use according to the invention, the patient possesses at least one tumor or is to be protected from a tumor which is of the same type as that from which the tumor cell is derived. Methods for determining the tumor type are disclosed in pathology textbooks.

This means that vaccinating with the tumor cell induces an immune response in the patient against the same tumor type as that of the tumor cell. This immune response is then as a rule based on antigens which are specific for the tumor (what are termed tumor antigens, see below) and which-are presented in the MHC complexes of the tumor cell which is used for the vaccination.

However, the immune response can also be based on recognizing other molecules, e.g. tissue-specific differentiation antigens or glycoproteins/glyco-peptides.

However, the invention also includes the tumor cell which is used in accordance with the invention being of a different type from the tumor which is to be treated in the patient or which is to be prevented. In this case, the immune cells recognize the proteins/peptides on the tumor cell surface which are also present on the surface of the tumor. These proteins/peptides may or may not be bound to MHC molecules.

According to another preferred embodiment, the tumor cell is derived from a primary tumor or a metastasis.

The tumor cell is preferably derived from a tumor which is selected from the group comprising melanoma, mammary carcinoma, colon carcinoma, ovarian carcinoma, lymphoma, leukemia, prostate carcinoma, lung carcinoma, bronchial carcinoma and pancreatic carcinoma.

According to another preferred embodiment, the tumor cell which is used in accordance with the invention expresses at least one tumor antigen which is characteristic for the given tumor, for example a cellular or viral tumor antigen. This tumor antigen is preferably recognized by the immune system, leading to activation of the immune system and then to the treatment of, or prevention in, the patient.

According to a particularly preferred embodiment, the tumor antigen is selected from the group comprising MART, Her2neu, tyrosinase, tyrosinase-related proteins (TRP), MART1/MelanA, Ny-ESO-1, CEA1, CEA2, CEA3, α-feto protein, MAGE X2, BAGE, GAGE1, GAGE2, GAGE3, GAGE4, GAGE5, GAGE6, GAGE7, GAGE7a, GAGE8, MAGE A4, MAGE A5, MAGE A8, MAGE A9, MAGE A10, MAGE A11; MAGE A12, MAGE1, MAGE2, MAGE3, MAGE3b, MAGE4a, MAGE4b, MAGE5, MAGE5a, MAGE5b, MAGE6, MAGE7, MAGE8, MAGE9, PAGE1, PAGE4, CAMEL, PRAME, LAGE1, gp100, Her2neu, ras, p53, E6, E7 and SV40 large and small T antigens.

According to a very particularly preferred embodiment, the tumor cell is derived from a melanoma and the tumor antigen is selected from the group comprising tyrosinase, MART1/MelanA, Ny-ESO-1, MAGE3 and gp100.

According to another preferred embodiment, the tumor cell expresses at least one cytokine and/or chemokine, preferably selected from the group comprising GM-CSF, G-CSF, IL1, IL2, IL3, IL4, IL5, IL6, IL7, IL8, IL9, IL10, IL11, IL12, IL13, IL14, IL15, IL16, IL17, IL18, IL19, IL20, IL21, IL22, IFNα, IFNβ, IFNγ, Flt3 L, Flt3, TNFα, RANTES, MIP1α, MIP1β, MIP1γ, MIPδ, MIP2, MIP2α, MIP2β, MIP3_{α}, MIP3β, MIP4, MIP5, MCP1, MCP1β, MCP2, MCP3, MCP4, MCP5, MCP6, 6cykine, Dcck1 and DCDF. Preferred cytokines/chemokines are GM-CSF, RANTES and/or MIP1α.

According to another preferred embodiment, the tumor cell which is used in accordance with the invention can also express a fusion protein formed from the abovementioned polypeptides. In addition, the invention also includes the tumor cell expressing functional variants of the abovementioned polypeptides, with functional variants being characterized by the fact that they essentially possess the same biological activity as said polypeptides. The prior art discloses tests for detecting the respective polypeptides or for measuring their respective activities.

According to a very particularly preferred embodiment, the tumor cell which is used in accordance with the invention expresses B7.2 and GM-CSF. As can be seen from Example 2, combining these two polypeptides results in the tumor cell having a surprisingly high activity in patients.

It was shown in a mouse model that, even within the context of an allogenic vaccination without any congruence in the MHC molecules, melanoma cells which have been altered recombinantly such that they express the two polypeptides B7.2 and GMCSF are more effective than tumor cells which only express GMCSF.

In detail, mice were given an intravenous injection of live unaltered tumor cells in order to provoke the formation of lung metastases. The animals were then vaccinated twice with irradiated tumor cells which were either unaltered, expressed GMCSF or expressed B7.2 and GMCSF. It was shown that the two peptides had a synergic effect both in the autologous vaccination situation, i.e. tumor cells (vaccine) from the same animal, and in the allogenic situation without any congruence in the MHC molecules, i.e. tumor cells from an animal possessing a different MHC type. While this result is known both in the case of the autologous situation and in the case of an allogenic situation in which there is partial congruence between the MHC molecules, it is completely surprising in the case of an allogenic situation in which there is no congruence between the MHC molecules.

An effect of this nature is not compatible with the previously known mode of action of costimulatory molecules. According to the generally accepted state of knowledge, antigen-specific T cells are activated by way of two signal pathways: in the first place, the antigen fragment, which has been loaded onto endogenous MHC, is presented to the T cell receptor; in the second place, a receptor-ligand bond is formed between the B7.2 on the antigen-presenting cell and CD28 on the T cell. Both signals are necessary for the T cell to be activated (two signal model, see, for example, Bretscher P (1992) Immunol Today 1992 February; 13(2): 74-6).

However, according to prevailing opinion, it is only possible for an interaction to take place between the tumor antigen-specific. T cell receptor and the MHC molecule when both cells carry the same MHC type, i.e. are derived from the same patient. Such an interaction is thus far unknown, or has not been demonstrated, in the allogenic case where there is no congruence in the MHC molecules. For this reason, the synergic effect which is demonstrated in the present experiments is completely surprising.

The synergic effect of the two molecules, as shown in the above example, must therefore use another route of T cell activation. Without being bound to any theories, it is known that NK cells (natural killer cells) also carry CD28, that is the receptor for B7.2 (Nandi D et al. (1994) J. Immunol. 1;152(7): 3361-9; Amakata Y et al (2001) Clin Exp Immunol. 124(2): 214-22; Martin-Fontecha A et al. (1999) J. Immunol. 15;162(10): 5910-6; Yeh KY et al. (1995) Cell Immunol. 15;165(2): 217-24).

NK cells are also activated by interaction with B7.2. However, it is generally assumed that this interaction only results in an increase in cytokine production (see Nandi D above, Amakata Y above, Marin-Fontecha A above and Yeh KY above), and not in any increase in lytic activity. It was surprisingly possible, within the context of the present invention, to demonstrate this increase in the lytic activity of NK cells. In the case of an allogenic vaccination without any congruence in the MHC molecules, this can result both in an increase in the lysis of the vaccine cells, and consequently in the release of antigens, which can subsequently be more effectively taken up by the patient's antigen-presenting cells and presented to T cells, and in the previously observed secretion of various cytokines which stimulate T cells and antigen-presenting cells. This effect is also surprising since it has thus far always been denied, in the prior art, that costimulatory molecules have a stimulating effect in the case of allogenic vaccines without any congruence in the MHC molecules (Wu TC et al (1995) J Exp Med. 1;182(5): 1415-21; Huang AY et al (1996) J Exp Med. 1;183(3): 769-76) since, according to these experiments, the expression of B7 had no effect in conjunction with a first vaccination.

In addition, the fact that anti-allogen-reactive T cells, which recognize foreign MHC molecules, are more strongly activated by way of B7.2 on the allogenic vaccine cells probably contributes to the effect of the allogenic vaccine. As a result, the vaccine cells are once again lyzed more efficiently and made available as an antigen source for antigen-presenting cells. In this connection, the B7.2 produces an adjuvant effect.

This means that, in contrast to an autologous vaccine or an allogenic vaccine having at least partial congruence in the MHC molecules, the immunostimulatory effect is primarily mediated by T cells, the immunostimulatory effect in the case of an allogenic vaccine without any congruence in the MHC molecules is mediated by NK cells and allospecific T cells, with the allospecific T cells presumably being unable to specifically recognize the body's own tumor cells (Schnurr M et al. (2002) Cancer Res. 15;62(8): 2347-52).

According to a preferred embodiment, the tumor cell which is used in accordance with the invention harbors one or more vector(s) which bring(s) about the expression of one or more of the above-defined polypeptides.

This/these vector(s) thus has/have the effect that the polypeptide is expressed in the tumor cell. This can take place in a variety of ways. In the first place, the vector can comprise control sequences which bring about the expression of the polypeptide using the endogenous gene for the polypeptide. Such vectors are known in the prior art.

According to a particularly preferred embodiment, the vector comprises nucleic acid sequences which encode the abovementioned polypeptides. The respective nucleic acid sequences are known in the prior art and can be obtained, for example, from the above-listed literature citations. The prior art discloses in principle how to construct a vector such that a polypeptide can be expressed. (Sambrook J. et al. (1989) Molecular Cloning: A Laboratory Manual, 2^{nd} ed., Cold Spring Harbor: Cold Spring Harbor Laboratory).

According to a particularly preferred embodiment, the vector is of nonviral origin, for example current expression plasmids, or of viral origin, preferably being derived from AAV, HSV, retrovirus, lentivirus, adenovirus or SV40.

According to another preferred embodiment, the vector is present episomally or is integrated into the genome of the cell.

This as a rule depends on the vector itself. The prior art discloses how the vectors are to be constructed such that they are either present episomally or are integrated into the genome of the cell (Sambrook J, see above).

According to a very particularly preferred embodiment, the vector is derived from AAV. AAV vectors, and vectors which are derived from them, are known in the prior art (see, for example, WO 00/47757 and WO 02/20748).

According to a very particularly preferred embodiment, the AAV vector which is harbored by the tumor cell which is used in accordance with the invention is present as a concatemer in the AAV-S1 acceptor site.

This would have the advantage that appropriate AAV vectors would be integrated at a site in the genome which is known not to result in any negative positional effects. In addition, such insertions into the genome are more stable when present as a concatemer and, as a result of the increased number of copies, exhibit stronger expression of the transgenes. The prior art discloses how to detect the presence of concatemers. This can be done, for example, by means of quantitative PCR/Taqman PCR, quantitative in-situ hybridization or amplifying the insertion site and then determining the length of the amplified DNA fragments.

According to another preferred embodiment, the expression of the polypeptide is controlled by a constitutive promoter, for example the CMV promoter (Vincen et al (1990) Vaccine 90, 353, the SV40 promoter (Samulski et al (1989) J Virol 63, 3822) or the retroviral LTR promoter (Lipkowski et al (1988) Mol Cell Biol 8, 3988), an inducible promoter, for example the tet promoter, and/or a tissue-specific promoter, for example the elongation factor promoter, the Ig promoter or the IL2/NFAT promoter.

The promoters can control the expression either by controlling the expression of the polypeptide using the endogenous genes or by encoding the expression of the polypeptides using the nucleic acid sequences which are contained in the vector. The prior art discloses a large number of promoters which can be used in accordance with the invention. (Sambrook J see above).

According to a very particularly preferred embodiment, the tumor cell which is used in accordance with the invention is proliferation-incompetent, for example as a result of being irradiated or chemically inactivated.

According to the invention, “proliferation incompetent” is understood as meaning that the tumor cell is no longer able to proliferate.

For example, it is known that gamma irradiation with 25-100 Gy makes tumor cells proliferation incompetent (see, for example, WO 97/32988). The addition of 40 μg/ml of mitomycin/ml can be used, for example, to effect a chemical inactivation.

The vaccine which is produced by the use according to the invention is employed to induce an immune response in the patient. To achieve this, it is not necessary, in some cases, for the pharmaceutical to additionally comprise an adjuvant. In this connection, an adjuvant is defined, within the context of the invention, as being a compound which is able to augment the induction of an immune reaction.

However, in many cases it is necessary for the pharmaceutical to additionally comprise an adjuvant. According to a preferred embodiment, the pharmaceutical consequently comprises an adjuvant, preferably those adjuvants which act as Toll-like receptor agonists. CpG oligonucleotides are examples of these. CpG oligonucleotides are oligonucleotides which contain at least one CpG motif (see, for example, Wagner H (2001) Immunity 14, 499-502).

According to another embodiment, the adjuvant is derived from Calmette-Guerin bacillus cell wall skeleton (BCG-CWS). BCG-CWS is known to be a ligand of Toll-like receptors 2 and 4 and to be able to induce the differentiation of immune cells (Matsumoto M et al (2001) Int Immunopharmacol 1, 8, 1559-69).

According to another embodiment of the invention, the adjuvant is a superantigen. Superantigens are antigens which bind directly to T cell rectors and MHC molecules and bring about direct activation of the T cells. Superantigens are known to be also able to have an adjuvant effect (see, for example, Okamoto S et al (2001) Infect. Immun. 69, 11, 6633-42). Examples of known superantigens are Staphylococcus aureus enterotoxins A, B, C, D and E (SEA, SEB, SEC, SED, SEE), Staphylococcal aureus toxic shock syndrome toxin 1 (TSST-1), staphylococcal exfoliating toxin and streptococcal pyrogenic exotoxins.

According to another embodiment according to the invention, the adjuvant is an agent which inhibits the CTLA-4 signal effect.

According to another preferred embodiment, the pharmaceutical comprises suitable additives and/or binding agents. In this connection, the additive or binding agent preferably comprises from 0.3 to approx. 4 M, preferably from 0.4 to approx. 0.3 M, in particular from approx. 0.5 to 2 M, very particularly preferably from approx. 1 to approx. 2 M of a salt at a pH of approx. 7.3-7.45, in particular 7.4.

The salt is preferably an alkali metal salt or an alkaline earth metal salt, in particular a halide or a phosphate, in particular an alkali metal halide, very particularly preferably NaCl or KCl.

According to another preferred embodiment, the pH is adjusted using a buffer, for example using a phosphate buffer, a Tris buffer, a HEPES buffer or a MOPS buffer.

Within the context of the uses according to the invention, the tumor cells are administered in quantities of preferably at least 1×10⁵, preferably 1×10⁶, in particular 1×10⁷ cells per dose. These quantities apply both to the prophylactic vaccination and the therapeutic vaccination. In the case of the prophylactic vaccination, the cells are preferably administered at least twice, particularly preferably at least three times, at intervals of at least 2 weeks, preferably 4 weeks, in particular 8 weeks.

The administration is as a rule subcutaneous, intracutaneous or intranodal.

According to a preferred embodiment, the tumor cell is derived from an individual of the same species as the patient.

According to a preferred embodiment of the invention, the patient is a mammal, preferably a human being.

According to a preferred embodiment, the vaccine which is produced within the context of the use according to the invention brings about an activation of the lytic activity of NK cells.

The prior art discloses methods for measuring the activation of the lytic activity of NK cells (Current Protocols in Immunology, chapter 7.18, 7.7.4-7.7.5, 7.7.8-7.7.10, John Wiley & Sons, 2002).

The invention furthermore relates to the use of a costimulatory polypeptide-expressing tumor cell for producing a vaccine for increasing the lytic activity of NK cells when treating or preventing a tumor in a patient who is allogenic with respect to the tumor cell.

As already explained above, the term “allogenic” means, within the context of the present invention, that two individuals (or an individual and the cell which is used for the vaccination) differ in regard to their antigens. This means, as a rule but not necessarily, that they differ in regard to their HLA antigens. In this connection, the possibility of the two individuals being partially congruent in regard to the HLA genes is expressly included. Complete congruence, or no congruence, in regard to the HLA genes is also included. In the former case, at least one further antigen then differs between the individuals (or the cell and the patient).

Within the context of the invention, it has been surprisingly found, as explained above, that the activation, which is brought about by the costimulatory molecule, of the lytic activity of NK cells makes it possible to treat and/or prevent tumors. This makes it possible, for the first time, to treat a group of patients with an allogenic vaccine, which comprises a costimulatory polypeptide-expressing tumor cell, without it being necessary to determine the HLA type of the patient and match the allogenic vaccine to the HLA type. It is consequently possible, for the first time, for all patients, irrespective of their HLA type, to be able to profit from an allogenic vaccine of this nature.

The same embodiment forms as described above within the context of the second use according to the invention apply to the costimulatory polypeptide, the tumor cell, the tumor, the vaccine and the patient. The invention also includes the possibility of the patient and tumor cell being partially congruent in regard to their HLA antigens.

The invention also relates to a method for treatment or prevention in a patient, in which method a therapeutically effective quantity of a costimulatory polypeptide-expressing tumor cell is administered to the patient, with the tumor cell and the patient not exhibiting any congruence in their MHC complexes.

The invention furthermore relates to a method for treatment or prevention in a patient, in which method a therapeutically effective quantity of a costimulatory polypeptide-expressing tumor cell is administered to the patient, resulting in an activation of the lytic activity of NK cells, with the tumor cell being allogenic with respect to the patient.

Within the context of the methods according to the invention, the same embodiments as described above within the context of the uses according to the invention apply to the costimulatory polypeptide, the tumor cell, the tumor, the vaccine and the patient.

The invention opens up completely new perspectives for the treatment of tumor patients.

The figures and the following examples are intended to explain the invention in more detail without restricting it.

### BRIEF DESCRIPTION OF THE FIGURES

FIG. 1 depicts the graphic analysis of an intracellular IFN-γ assay. Immunostaining was used to measure the secretion of IFN-γ from HLA-A2-positive donor T cells which had been stimulated with Mel29 tumor cells having HLA congruence (above) or with Mel 62 tumor cells without HLA congruence (below). In both cases, B7.2-transduced tumor cells were compared with untransduced tumor cells.

FIG. 2 depicts the average number of lung metastases per group in connection with a therapeutic vaccination in a lung metastasis model.

FIG. 3 depicts the average lung weight in the groups in connection with a therapeutic vaccination in a lung metastasis model.

FIG. 4 depicts the graphic analysis of a chromium release experiment in which the release of ⁵¹Cr from correspondingly labeled melanoma cells was measured at different ratios of effector cells (NK cells) and target cells (melanoma cells). The NK cells were obtained from the incubation of PBLs with untransduced melanoma cells or B7.2-transduced melanoma cells.

FIG. 5 depicts the graphic analysis of an experiment in which untransduced melanoma cells, or melanoma cells which had been transduced, by means of rAAV, with B7.2, or with B7.2 and GM-CSF, were incubated with PBLs for 5 days and their proliferation was measured by the incorporation of ³H-Tdr.

FIG. 6 depicts the graphic analysis of a mouse vaccination experiment in which tumors were preimplanted and in which autologous and allogenic vaccination strategies were compared with each other. Cell lines used for the vaccination, and transgenes expressed by them, are plotted on the X axis; the relative tumor load in % was evaluated.

### EXAMPLES

### Example 1

Preparing T lymphocytes specific for melanoma antigens Cells/materials:
Mel 29: melanoma patient-derived melanoma cell line, HLA-A2.01-positive, untransduced or B7.2/GMCSF-transduced;
Mel 62: melanoma patient-derived melanoma cell line, HLA-A2.01-negative, untransduced or B7.2/GMCSF-transduced;
T2 cells: TAP-deficient lymphoblastoid cells; they were loaded with a complex composed of HLA-A2.01-restricted melanoma peptides;
peripheral blood lymphocytes (PBLS) from a healthy donor, HLA-A2.01 positive;
rAAV B7.2
   A. Primary Stimulation of the PBMCs

Melanoma cells are sown, at a density of 3.8×10⁴ cells in in each case 1 ml of medium (DMEM containing 10% FCS, 2 mM L-glutamine, 1× antibiotic/antimycotic, 1× MEM vitamins; Gibco-BRL) in three wells of a 24-well plate. On the following day, the cells are irradiated with 100 Gy and infected with 20 μL of rAAVB7.2/GM-CSF virus (corresponds to an MOI of 84). After having been incubated at 37° C. and 5% CO₂ for, 48 h, the culture supernatant is aspirated from the transduced melanoma cells and 2.5×10⁶ PBMCS from an HLA-A2-positive donor are added.

B. Restimulation of the PBMCs

After 1 week, the PBMCs are restimulated with peptide-loaded PBMCs. For this, 1.5×10⁷ PBMCs from the same donor are treated with 10 μg/μl MARTₘᵤₜ peptide having the sequence ELAGIGILTV and incubated at 37° C. and 5% CO₂ for 4 h. After that, the cells are diluted with T cell medium containing 10% human serum and 0.4 U/ml IL-2 (Boehringer Ingelheim) up to a final concentration of the peptides of 0.5 μg/ml and a final concentration of the cells of 3-4×10⁵/ml. 1 ml of culture supernatant is aspirated from the primary stimulation mixture and 3-4×10⁵ peptide-loaded PBMCs are added in 1 ml.

Two weeks after primary stimulation, the cells are restimulated as described above. Six days later, the cells are restimulated for the third time. For this purpose, T2 cells (TAP-deficient B cell lymphoma) are loaded with peptide, as described above, after which they are irradiated with 100 Gy; 1×10⁵ cells are then added to the stimulation mixture. The restimulation takes place without IL-2.

C. Using Intracellular FACS Staining to Analyze the Production of IFN-γ in CDB+ T cells

On the day after the third restimulation, an intracellular IFN-γ staining is carried out. The culture of the cells is continued and the assay is repeated one week later.

In order to stimulate the production of IFN-γ in the T cells, they are incubated with peptide-loaded antigen-presenting cells. T2 cells are adjusted to a concentration of 1×10⁶ cells/ml and treated with 10 μg of Martₘᵤₜ peptide or HIV peptide/ml. 50 μl of this mixture are sown, per well, in a 96-well round-bottom plate and incubated overnight at 37° C. and 5% CO₂.

An aliquot of the restimulated PBMCs is harvested, resuspended in assay medium (RPMI 1640, Gibco BRL, Cat# 21875-034; 1 mM Na pyruvate; 2 mM L-glutamine; 1× MEM nonessential amino acids; 50 μg of gentamicin/ml; freshly treated with 10% human serum; 0.8 U of IL-2/ml) and adjusted to 1×10⁷/ml. 50 μl of cell suspension from this mixture are added to the peptide-loaded T2 cells.

In order to improve the interaction of the cells, the plate is centrifuged for 1 min at 1200 rpm and 4° C.

Following incubation at 37° C. and 5% CO₂ for 1 h, 10 μl of a monensin solution (3 mM monensin in 100% ethanol, Sigma) diluted 1:100 in RPMI are added. The cells are incubated at 37° C. and 5% CO₂ for 5 h and then centrifuged at 1200 rpm and 4° C. for 2 min; the supernatant is then removed. After that, 100. PI of cytofix/cytoperm solution (Pharmingen) are added per well; the cells are then thoroughly resuspended by being pipetted up and down several times and incubated on ice for 20 min. The cells are harvested by being centrifuged at 1200, rpm for. 2 min; the supernatant is tipped off and the cells are resuspended by being gently vortexed. After having been washed twice with in each case 100 μl of perm/wash solution (Pharmingen; diluted 1:10 in double distilled water), and centrifuged (as described above), the cells are stained. In each case 20 μl of the following antibodies, diluted 1/50 in 1× perm/wash solution, are added: anti-IFN-γ FITC (Caltag, Cat# MHCIFG01), anti-CD8-PE (Becton Dickinson, Cat# 30325×) and anti-CD4 cychrome (Becton Dickinson, Cat# 30158×); the plate is then vortexed briefly and incubated on ice for 30 min. The cells are washed 2× with in each case 100 μl of perm/wash solution, after which the cell pellets are in each case resuspended in 150 μl of PBS/0.5% BSA and transferred to micronic tubes. After that, the samples are measured in the FACS.

While a specific intracellular IFN—Y staining was found in T cells which had been originally stimulated with Mel62-B7.2/GM-CSF, stimulation with untransduced Mel62 had no effect. The production of IFN-γ was peptide-specific since HIV peptide-loaded T2 cells were without effect. The result was confirmed one week later.

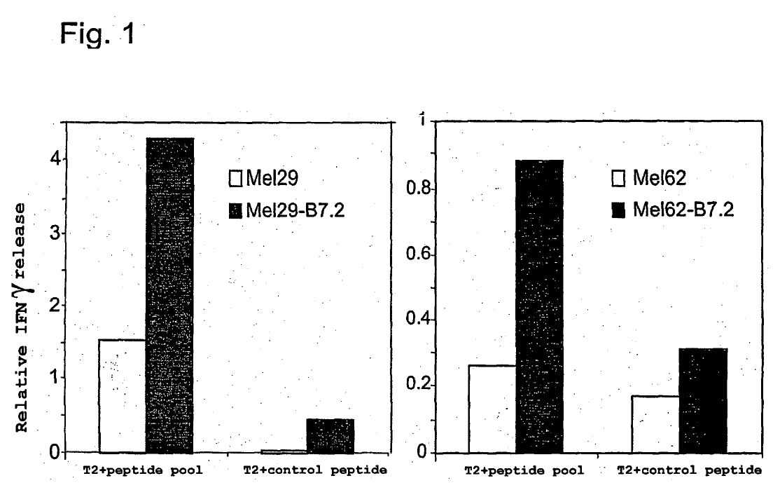

FIG. 1 clearly shows that the activation of the T cells is not dependent on whether the donor T cells and the stimulation cells (Mel29 or Mel62) are congruent in the HLA haplotypes or not. In both cases, it was clear that the T cells were activated substantially more powerfully by B7.2 transduced melanoma cells than by untransduced melanoma cells.

In other experiments using control vectors, it was shown that this effect depended on the expression of B7.2. The antigens which were tested were Mart1/MelanA, gp100 and tyrosinase (peptide pool).

The specificity of these melanoma-derived antigens was demonstrated in comparison to a control peptide derived from HIV gp120. Greater numbers of specific T cells were observed when an allogenic stimulation involving HLA-A2 congruence (Mel29, FIG. 1, top) than when an allogenic stimulation without any HLA congruence (Mel62, FIG. 1, bottom) was carried out. In this experimental system, the antigenic signal comes from tumor antigens which are expressed endogenously by the melanoma cell and are presented by an MHC molecule which is common to both the stimulating cell and the T cell (HLA-A2 congruence). In the situation without any HLA congruence, the activation of a T, cell depends on the presentation of the melanoma antigens by induced antigen-presenting cells (APCs), such as dendritic cells (cross-priming).

### Example 2

Comparison of Autologous/Allogenic Vaccination for Treating Preimplanted Tumors in an Animal Model

Summary of the Experiment:

Species/strain: mouse, C3H/He (H-2k)
Age, sex: 6-10 weeks of age, female
Body weight: 30 g
Assay components: B16F10-HEL-wt cells (H-2b), transfected with B7.2and/or GM-CSF pAAV plasmid
   K-1735-HEL (H-2k), transduced with rAAV-B7.2/GM-CSF
Dose, route: s.c., 3×10⁵ cells
Aim of the experiment: Prevention/retardation of the tumor colonization or the tumor growth in connection with B7.2/GM-CSF vaccination as compared with control vaccines.
Design: 1.2×10⁵ unmodified K1735-HEL cells (except in the case of experiment TV19: 1.0×10⁵ cells) were injected i.v. into C3H/He mice. Four and 11 days later, the animals were immunized s.c. with genetically modified (Shastri, University of CA, Berkeley, CA, USA) and irradiated variants of the allogenic line B16F10-HEL and the syngenic (corresponding to an autologous) line K1735-HEL (dose: 3×10⁵ cells). The development of tumor metastases in the lung was examined by means of dissecting and weighing the lungs on day 21 after the challenge.
   Preparing HEL-Expressing Tumor Cells:

The expression vector pcDNA3neo-HEL was cloned for the purpose of preparing stable transfectants of the melanoma cell lines. B16F10 (Prof. Isaiah J. Fidler, MD Anderson Cancer Centre, Texas, USA) and. K-1735 (Dr Souberbielle, King's College, London). To do this, the HEL gene was excised from the vector pcDNA1-HEL (Shastri, University of CA, Berkeley, Calif., USA) and ligated into the expression vector pcDNA3neo (Invitrogen, Carlsbad, Calif., USA), which contains a gene for resistance to neomycin which is used for selecting positive clones.

Lippfectamine® (# 11668, Invitrogen, Carlsbad, Calif., USA) was used to transfect B16F10 and K-1735 cells on 15 cm culture dishes. Positive cells were selected using G418-containing medium (800 μg/ml). After 2-3 weeks, individual clones were picked and expanded. RT-PCR and Western blotting were used to examine the clones for expression of the transgene. The two clones having the best expression rates were selected for vaccination experiments.

RT-PCR:

RNA was prepared from 2-5×10⁶ cells using QIAshredder columns (# 79654, QIAgen®, Hilden) and the RNeasy kit (# 74104, QIAgen®, Hilden).

DNA (e.g. episomal plasmid DNA) was removed using RNAse-free DNAse. (# 776785, Roche®, Basle).

RNA was transcribed into cDNA using the Gene Amp RNA PCR core kit (Applied Biosystems for Roche®, # N 808-0143, Foster City, Calif., USA).

PCR on HEL and β-actin was carried out using the Taq-Mastermix kit (# 1007 544, QIAgen, Hilden) and the following primers:

| | |
|---|---|
| HEL-up | (5′-AGG TCT TTG CTA ATC TTG GTG C-3′) |
| HEL-down | (5′-GGC AGC CTC TGA TCC ACG-3′) |
| mu β- | (5′-GAT CCT GAC CGA GCG TGG CTA C-3′) |
| actin-up | |
| mu β- | (5′-CAA CGT CAC ACT TCA TGA TGG AAT TG-3′) |
| actin- | |
| down | |

The amplified HEL fragment had a length of 430 bp, while the amplified fragment of murine β-actin had a length of 290 bp.

Western Blotting:

Cells were lysed with cell lysis buffer
Lysates were loaded onto 12% polyacrylamide gels using DTT-containing loading buffer.
Hen egg lysocyme (Sigma®, #L-6876, Deisenhofen) was used as the standard.
Transfer to nitrocellulose membranes was effected using a semi-dry transfer system.
The blocking of unwanted background, as well as antibody incubation steps, were carried out in 5% milk powder in TBST (Tris-buffered saline, 0.01% Tween (TBST))
Antibody: biotinylated anti-HEL at a dilution of 1:200 (RDI, #RDI-lyszym-BT, Flanders, N.J.).
Streptavidin-HRP: used at a dilution of 1:5000 (Sigma®, #S-5512, Deisenhofen)
Super Signal (Pierce®>#34080, Rockford, Ill., USA) was used as substrate for the chemiluminescence reaction. X-ray films were exposed to the blots for between 30 seconds and 1 hour.
   Preparing B7.2 and GM-CSF-Expressing K1735-HEL Vaccine Cells:

K1735-HEL cells which express murine B7.2, GM-CSF or both molecules were produced by transducing them with recombinant adenoassociated virus (AAV). The plasmids pAAV-muGMCSF and pAAV-muB7.2 were cloned for this purpose: the cDNA for GM-CSF and B7.2 were cloned into the vector pCI (Promega, Madison, Wis., USA), which provides a CMV promoter and an SV40 3′-untranslated region. To obtain the pAAV-GM-CSF plasmid, two expression cassettes, containing GM-CSF together with the CMV promoter and the SV40 pA site, were ligated in tandem into the basic pAAV plasmid vector (see WO 00/47757, Example 4). In order to secure the ideal size for the virus packaging, a further 400 bp from pUC19 (bp 1516-1910) were also integrated into the vector.

In order to obtain the pAAV-B7.2 plasmid, the expression cassette, containing B7.2, the CMV promoter and the SV40 pA site, was ligated into the basic pAAV plasmid vector. A further 700 bp from pUC19 (bp 1201-1910) were required in order to generate the optimal size for the vector.

Producing Recombinant AAV:

Hela T cells were transfected simultaneously with 2 plasmids by means of calcium phosphate coprecipitation: i.e. with the vector plasmid pAAV-muGM-CSF or pAAV-muB7.2 and the AAV helper plasmid pUC“rep/cap”(RBS)□37, which carries the AAV genes for AAV2 rep and cap (see WO 00/47757).

After 2 days, the cells were infected with adenovirus. Four days later, the cells were lysed by being subjected 3 times to a freezing-thawing cycle at −20° C. and 37° C. Supernatants were removed, debris was centrifuged off and contaminating adenovirus was inactivated by treating it at 60° C. for 30 minutes. More detailed information in regard to preparing AAV can be obtained from the patent publication WO 00/47757.

K-1735-HEL cells were irradiated (100 Gy) and infected with the AAV. In order to obtain optimal GM-CSF expression (200-300 ng/10⁶ cells in 48 h), approx. 700 μl of the rAAV-muGMCSF virus were normally used for 5×10⁶ cells. In order to obtain optimal B7.2 expression (70%-96%), approx. 4 ml of the rAAV-muB7.2 virus were required for 5×10⁶ cells. After 2 days, the cells were harvested, frozen (FCS, 10% DMSO) and stored in liquid nitrogen.

In order to prepare the cells for administration to mice, they were thawed in a 37° C. waterbath, washed three times in PBS and adjusted to the correct cell count (3×10⁵ cells per dose in PBS).

Preparing B7.2 and GM-CSF-expressing B16F10-HEL vaccination cells: It is not possible to efficiently transduce B16F10 cells with rAAV. For this reason, in order to generate vaccine cells for allogenic vaccination experiments, the transfection was carried out using the liposome Polyfect (QIAgen®, Hilden). In order to prepare transient transfectants, the melanoma cell line B16F10-HEL was transfected with the vectors pAAV-muGMCSF and pAAV-muB7.2 singly or in combination. The cells were sown in cell culture flasks (1.66×10⁶ per T75) and transfected on the following day in accordance with the manufacturer's instructions. 9 μg of plasmid were used for expressing B7.2, while 8 μg of plasmid were used for expressing GMCSF and 8 μg of B7.2 plasmid and 6 μg of GMCSF plasmid were used for the combination of the molecules. On the day after that, the medium was changed. On day 2, the cells were harvested by trypsinization, irradiated (100 Gy) and frozen down. The cells were stored in liquid nitrogen. Wild-type tumor cells were used as the negative control since, because of toxic side-effects, it was not possible to transfect with a basic pAAV vector.

The expression of the transgenes was measured by means of ELISA in the case of GM-CSF and by means of flow cytometry in the case of B7.2.

In order to prepare cells for injection into mice, they were thawed in a 37° C. waterbath, washed three times in PBS and adjusted to the correct cell count (3×10⁵ cells per dose in PBS).

Detecting the Expression of GM-CSF and B7.2:

Secreted GM-CSF was determined in the supernatant from transduced or transfected cells 48 h after sowing. The Pharmingen (San Diego, USA) OptEIA mouse GM-CSF enzyme-linked immunosorbent assay (ELISA) kit was used. B7.2 expression was measured by means of flow cytometry using the antibody GL1 (Pharmingen, Heidelberg).

Analysis of the Lung Metastases:

The mice were sacrificed by means of cervical dislocation on day 21 after the challenge. Immediately after that, the lungs were removed, weighed on an analytical balance and then fixed in Bouin's solution (85% picric acid, 10% formaldehyde, 5% glacial acetic acid). The number of metastasis nodes was determined under a microscope by counting them.

Evaluation

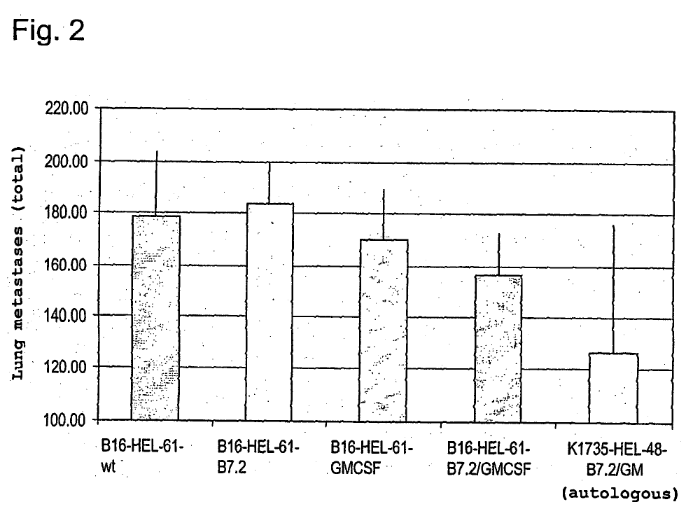

In the: mouse animal models which were investigated, it was observed that the allogenic vaccination mixture functioned just as well as the autologous mixture when treating preimplanted tumors. FIG. 2 shows that the B16 B7.2/GMCSF (allogenic) group does not differ statistically from the K1735 B7.2/GMCSF (autologous) group. Furthermore, the coexpression of B7.2 and GMCSF shows a synergic effect.

This is of particular importance with regard to the effect of B7.2 in connection with allogenic vaccination. As has, previously been explained, it is not possible for B7.2 to have an effect in allogenic vaccination, without any congruence in the MHC molecules, as the result of direct activation of antigen-specific T cells. This example demonstrates that an effect does nevertheless occur, but only in combination with another immunostimulatory, principle, in this case together with GMCSF. As has already been explained, the activation of natural killer cells, and the stimulation of anti-allogen-specific T cells, is regarded as being the mode of action.

Statistical evaluation of the experiment in Student's T test gave the following p values for the comparison of the groups which are in each case listed:

| | | p value |
|---|---|---|
| Experimental group 1: | Experimental group 2: | (T test): |
| B16-HEL B7.2 | B16-HEL B7.2-GMCSF | 0.007 |
| B16-HEL GMCSF | B16-HEL B7.2-GMCSF | 0.056 |
| B16-HEL B7.2-GMCSF | K17435-HEL B7.2-GMCSF | 0.84 |

This demonstrates the distinct superiority of the combination of B7.2 and GMCSF, as compared with using either of the two transgenes on its own, in connection with allogenic vaccination. The differences are statistically significant.

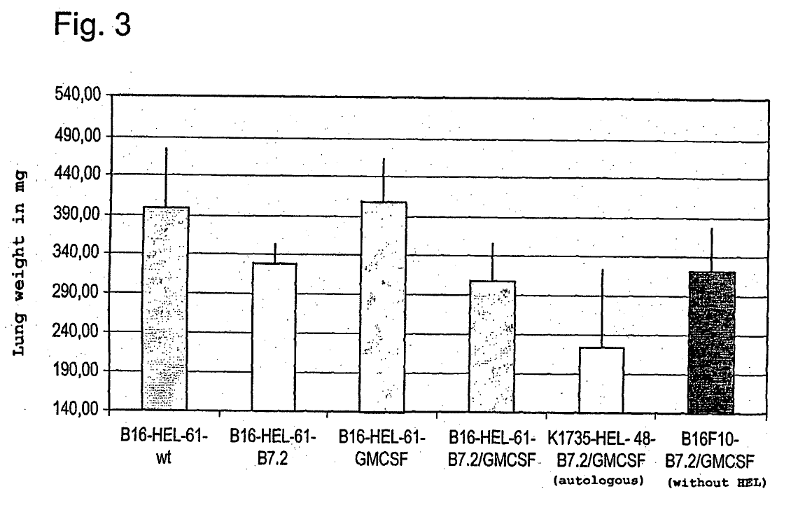

FIG. 3 shows that expression of B7.2 on its own has a positive effect. It shows that B7.2 has an influence on the development of an antitumor response even in an allogenic system without any congruence in the MHC molecules. However, combination with the cytokine GMCSF is of value since this then induces a more powerful immune response, which is manifested in a reduction in the tumor burden.

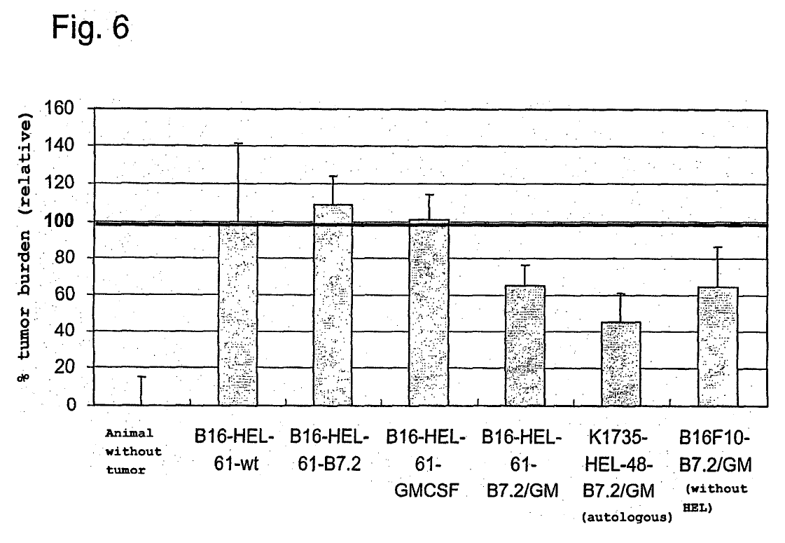

FIG. 6 depicts the joint evaluation of three similar but independent animal experiments. It demonstrates the clear superiority of the combination of B7.2 and GMCSF as compared with the molecules used singly. This synergic effect is particularly significant. The synergic effect of the molecules arises both in connection with autologous vaccination, where B7.2 is able to exert a direct effect on T cell activation, and in connection with allogenic vaccination without any congruence in the MHC molecules, where it is only possible to conceive of indirect effects by way of NK cells and alloreactive T cells. This underlines the interesting discovery that, despite previous opinion to the contrary, B7.2 exerts an immunostimulatory effect even in connection with allogenic vaccination. The results of the three experiments were weighted relative to each other by in each case setting the value of the average lung weight in the groups in which the animals did not undergo any manipulation (blank value) to be 0% and that of the group which were vaccinated with wild-type cells to be 100%. The lung weights of the individual, experimental groups were then weighted as percentages in relation to these values. A mean value was then calculated for all three experiments.

Statistical evaluation of these values in Student's T test gave the following p values for the comparisons of the groups which are in each case listed:

| Experimental group 1: | Experimental group 2: | p value (T test): |
|---|---|---|
| B16-HEL wt | K17435-HEL B7.2-GMCSF | 0.017 |
| B16-HEL B7.2 | B16-HEL B7.2-GMCSF | 0.014 |
| B16-HEL GMCSF | B16-HEL B7.2-GMCSF | 0.04 |

The differences are consequently statistically significant.

### Example 3

Effect of B7.2-Expressing Cells on NK Cell-Induced Cell Lysis

The experimental analysis of a postulated function of a melanoma vaccine in a human experimental setup involves several restrictions with regard to the methods which can be used and the parameters which can be investigated. Measuring an effect of cellularly produced GM-CSF is restricted to inducing the differentiation of monocytes into (pre)dendritic cells. The chemotactic effect is a parameter which can only be analyzed in vivo. On the other hand, the effect of B7.2 (CD86) can be investigated readily in T cell activation experiments and NK cell assays, as are described in the following examples

Summary of the Experiment

Cells/materials: Melanoma cell line, Mel 29 derived from a patient
   Peripheral blood lymphocytes (PBLs) obtained from a healthy donor
Test components: Mel 29 cells: untransduced or, transduced with B7.2
Aim of the experiment: Analysis of the effect of B7.2 on the NK cell-mediated lysis of melanoma cells
Design: PBLs were isolated fresh, purified through a Ficoll gradient and incubated overnight, at various E/T ratios, with 2×10^{3 51}Cr-labeled cells which were either untransduced or transduced with B7.2. The release of ⁵¹Cr by lysed cells was measured.
   Implementation
   Harvesting the Melanoma Cells and Labeling them with ⁵¹Chromium (Target Cells):

Cells in a T80 flask are detached and centrifuged down at 175×g for 5 min. The cell pellet is resuspended in 2-5 ml of assay medium (RPMI1640, Gibco, containing 5% FCS, 2 mM L-glutamine, 1 mM Na pyruvate, 1× nonessential amino acids, 50 μg of gentamicin/ml), and the cells are counted.

For the labeling with chromium, 100-200 μl of the cell suspension are transferred to a 1.5 ml Eppendorf tube (cell count: up to 1E+6). After 20-50 μl of ⁵¹Cr have been added, the cells are incubated at 37° C. and 5% CO₂ for from 45 min to 60 min. The cells are washed 2× and diluted to 2000 cells per 100 μl of medium.

Titrating the Effector Cells=Buffy Coat-Derived PBMNC, Freshly Isolated:

PBMNC are adjusted in assay medium to a cell count of 6.7E+6/ml; 150 μl of this suspension are pipetted into the first row of a 96-well round-bottom microtiter plate. A total of 6 titration steps are prepared from this in 1:3 dilution steps.

Pipetting the Effector and Target Cells Together:

In each case 100 μl of target cell suspension are pipetted into 100 μl of effector titration per well. In order to determine the spontaneous release of the cells, 100 μl of assay medium are added to 100 μl of target cell suspension. The cells are incubated at 37° C. and 5% CO₂ for approx. 16 h. After that, 100 μl of 2%. Triton X-100 are added to 100 μl of target cell, suspension in order to determine the maximum release.

Harvesting the Culture Supernatants and Measuring on a Top Count:

Fifty μl of the culture supernatants are transferred by pipette onto Luma plates and the plates are dried overnight. The dried plates are measured in a Top Count.

Results

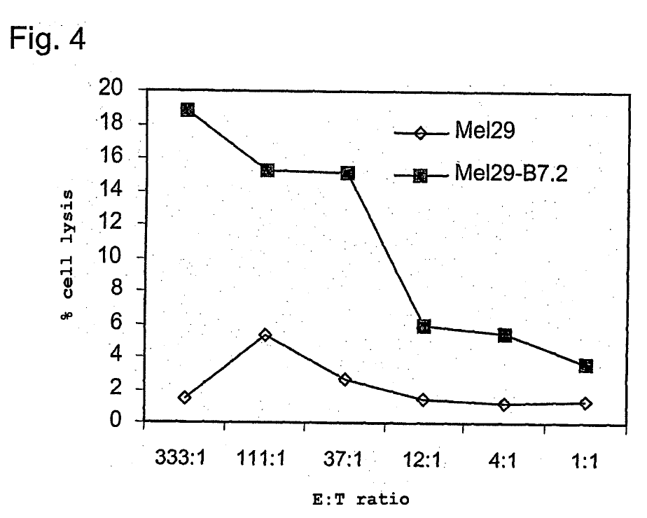

As can be seen in FIG. 0.4, the NK cell-mediated lysis of melanoma cells was markedly augmented by the Mel29 cells expressing B7.2. Similar results were obtained with a second melanoma cell line, i.e. Mel 62 (data not shown). In all, three out of five donors exhibited an increase in their NK cell activity which was comparable with that shown in FIG. 4. These results consequently demonstrate an increase in the NK cell-mediated lysis of tumor cells as a consequence of B7.2 being expressed on corresponding tumor cells. The consequences of such an increase in NK cell activity are (1) the release of cytokines, (2) the release of tumor antigen and (3) the activation of DC cells by the interaction of Cb40 and CD40L. As already stated, it is known that these consequences support the efficient activation of T cells against the tumor antigens which are derived from the melanoma cells.

### Example 4

Direct Activation of Human T Lymphocytes (Alloreactive T Cell Response)

Summary of the Experiment

Cells/materials: Melanoma cell lines Mel R3, Mel 29, Mel 66, Mel 68 and SkMel63 derived from a patient
   PBLs obtained from a healthy donor
Test components: Melanoma cells derived from a patient and transduced with:
   B7.2
   B7.2/GM-CSF
   Untransduced
Aim of the experiment: Analysis of T cell proliferation by means of ³H-Tdr incorporation
Design: 10⁴ melanoma cells were irradiated with 100 Gy and incubated, at 37° C. for 5 days, with 2.5×10⁵PBLs in 96-well plates. After that, ³H-Tdr (0.5 μCi) was added per well and the incubation, was continued for 18 hours. The incorporation of ³H-Tdr was determined by means of liquid scintillation counting.
   Implementation
   Stimulation:

Per well of a 96-well round-bottom microtiter plate, 1E+5 PBMNC/100 μl are incubated, at 37° C. and 5% CO₂ for 5 d, with 1E+4 irradiated (100 Gy) melanoma cells/100 μl (final volume, 200 μl). Culture medium RPMI1640 containing L-glutamine, with the following additions: 10% human serum, 1% L-glutamine, 1% sodium pyruvate, 1% nonessential amino acids and 0.1% gentamicin.

Incubating the Samples with ³H-Thymidine:

100 μl of the culture supernatant from each well are pipetted off; after that, 50 μl of culture medium containing 1 μCi of ³H-thymidine are added per well and the mixtures are subsequently incubated at 37° C. and 5% CO₂ for 16 h.

Harvesting the Samples and Measuring the Radioactivity:

The samples are precipitated on glass fiber filter mats using a semiautomatic sample harvesting appliance. The filters are dried for at least 1 h (or overnight) at 60° C. in a drying, oven. The dried filters are sealed in film and wetted with μ scintillation liquid. The μ radiation of the samples on the filters is then measured (cpm) in an instrument for measuring p radiation radioactivity.

Results

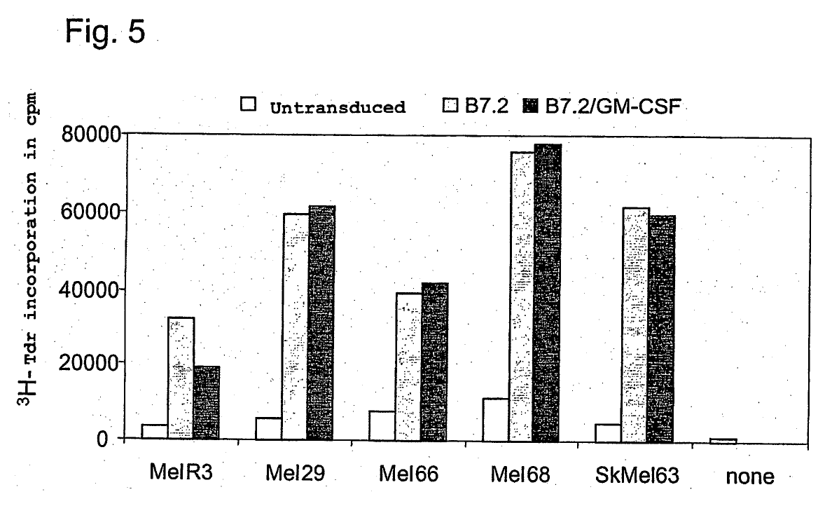

Melanoma cell lines which had been transduced with B7.2 on its own or with B7.2/GM-CSF gave rise to a markedly stronger proliferation of the T cells than did untransduced control cell lines (see FIG. 5. In this connection, the antigenic stimulus, is primarily supplied by the foreign MHC molecules. In a separate experimental series, melanoma cells giving increasing quantities of B7.2 expression were used to induce T cell proliferation. In this connection, it was found that the maximum T cell activation measured was obtained in the vicinity of a B7.2 expression rate of 30% positive cells (data not shown). In addition, it should be noted that transduction of melanoma cells with the control proteins GFP (green fluorescence protein) or lacZ (by infecting using rAAV) only had an insignificant effect on the T cell proliferation, with this effect being significantly less than the effect produced by B7.2 expression. This confirms that the observed increase in T cell proliferation depended on expression of B7.2.

In the present in-vitro experiment, it was not possible to observe any difference between B7.2-transduced cells and B7.2/GM-CSF-transduced cells (see FIG. 5). This can be explained by the fact that, while GM-CSF exerts chemotactic and activating effects on monocytes and DCs, it scarcely has any effects on the proliferation of T lymphocytes. Such positive effects resulting from GM-CSF expression have consequently to be investigated in in-vivo models.

### Example 5

Generating Melanoma Antigen-Specific T Lymphocytes

Summary of the Experiment

Cells/materials: Melanoma cell lines Mel 29 (HLA A2.01-positive) and Mel 62 (HLA A2.01-negative) derived from a patient
   T2 cells: TAP-deficient lympho-blastoid cells
   PBLs from a healthy, HLA A2.01-positive donor
Test components: Mel 62 and Mel 29 cells:
   Untransduced or transduced with B7.2/GM-CSF
Aim of the experiment: Induction of an immune response against peptide epitopes of known tumor antigens in an experimental setup with and without congruence of the MHC haplotypes (matched or mismatched) between melanoma cell lines and PBLs.
Design: 2.5×10⁶ Mel 29 cells or Mel 62 cells were incubated, at 37° C. for 7 days, with 5×10⁶ allogenic HLA-A2.01-positive PBLs (consequently matched) in T25 cell culture flasks. The T cells which were present in the PBLs were restimulated, over a period of 2 weeks, with 2.5×10⁶ T2 cells which had been loaded weekly with HLA A2.01-binding melanoma antigen peptides. (MART-1: AAGIGILTV, gp 100: YLEPGPVTA and tyrosinase: YMDGTMSQV). The T2-stimulated T cells were subsequently analyzed with regard to their specific reactivity. To do this, they were incubated with 10⁴ T2 cells which had been loaded for 6 hours with tumor antigens [(either with a peptide pool consisting of HLA A2-binding melanoma antigen peptides or consisting of control peptides (HIV-RT (?) HLA A2-binding peptides having the sequence ILKEPVHGV)]. In conclusion, IFNγ production was measured by means of antibody-mediated, intracellular, staining and subsequent flow cytometry.
   Results

Melanoma cells which had been transduced with rAAV-B7.2 induced, with a higher efficiency than did untransduced cells, the activation of T cell lines (Mel29 and Mel62) which specifically recognized known peptide epitopes of known melanoma antigens (see FIG. 6). In other experiments, it was possible to use control vectors to demonstrate that this effect depended on the expression of B7.2.

The antigens which were tested here comprised Mart1/MelanA, gp100 and tyrosinase (jointly in the peptide pool). The specificity for these melanoma-derived antigens was demonstrated by comparing with a control peptide derived from HIV-gp120. Increasing quantities of specific T cells were observed when either an HLA-A2-matched allogenic stimulation (with Me29, left-hand diagram in FIG. 6) or an HLA-mismatched stimulation (Mel62, right-hand diagram in FIG. 6) of the T cells was carried out.

In this experimental system, the antigen-acting signal is derived from endogenously expressed tumor antigens of a melanoma cell (stimulator cell) which are presented by MHC molecules on the cell surface. In the HLA-A2-matched situation, the T cells are also derived from an HLA-A2-positive donor and the T cell receptor on the T cells consequently recognizes the antigenic peptide directly as a result of the presentation of the antigen by a corresponding MHC molecule which is common to the stimulating cell and the donor of the T cells.

The surprising fact that a comparable activation of the T cells can be observed in the mismatched situation, and that T cells and stimulating cells do not possess any MHC molecules in common leads to the conclusion that the activation of the T cells has to take place indirectly. This must be the case since, in the mismatched situation, the T cells are no longer able to directly recognize the antigens which are presented by the MHC molecules on the stimulating cell. This indirect activation presumably depends on the presentation of the melanoma antigens by induced dendritic cells (also termed dross-priming).

It was consequently surprisingly possible to demonstrate that specific MHC-restricted T lymphocytes by direct contact between T cells and melanoma cells which express B7.2, are induced by means of indirect cross-priming by antigen-presenting cells (APCs).

1-30. (Cancelled)

31. A method for treating or preventing a tumor in a patient, said method comprising administering to a patient a tumor cell expressing a costimulatory polypeptide, wherein the tumor cell and the patient exhibit no congruence in their MHC molecules.

32. The method as claimed in claim 31, characterized in that the costimulatory polypeptide is selected from the group consisting of B7.1, B7.2, LIGHT, CD40L, Ox40, 4.1.BB, Icos L, SLAM, ICAM 1, LFA-3, B7.3, CD70, HSA, CD84, CD7, B7 RP-1 L, MAdCAM-1, VCAM-1, CS-1, CD82, CD30, CD120a, CD120b and TNFR-RP.

33. The method as claimed in claim 31, characterized in that the patient possesses at least one tumor, or is to be protected from a tumor, which is of the same type as that from which the tumor cell is derived.

34. The method as claimed in claim 31, characterized in that the tumor cell is derived from a primary tumor or a metastasis.

35. The method as claimed in claim 31, characterized in that the tumor cell is derived from a tumor which is selected from the group consisting of melanoma, mammary carcinoma, colon carcinoma, ovarian carcinoma, lymphoma, leukemia, prostate carcinoma, lung carcinoma, bronchial carcinoma and pancreatic carcinoma.

36. The method as claimed in claim 31, characterized in that the tumor cell expresses at least one tumor antigen which is characteristic for the respective tumor.

37. The method as claimed in claim 36, characterized in that the tumor antigen is selected from the group consisting of MART, Her2neu, tyrosinase, tyrosinase-related proteins (TRP), MART1/MelanA, Ny-ESO-1, CEA1, CEA2, CEA3, α-feto protein, MAGE X2, BAGE, GAGE1, GAGE2, GAGE3, GAGE4, GAGE5, GAGE6, GAGE7, GAGE7a, GAGE8, MAGE A4, MAGE A5, MAGE A8, MAGE A9, MAGE A10, MAGE A11, MAGE A12, MAGE1, MAGE2, MAGE3, MAGE3b, MAGE4a, MAGE4b, MAGE5, MAGE5a, MAGE5b, MAGE6, MAGE7, MAGE8, MAGE9, PAGE1, PAGE4, CAMEL, PRAME, LAGE1, gp100, ras, p53, E6, E7 and SV40 large and small T antigens.

38. The method as claimed in claim 36, characterized in that the tumor cell is derived from a melanoma and in that the tumor antigen is selected from the group consisting of tyrosinase, MART1/MelanA, Ny-ESO-1, MAGE3 and gp100.

39. The method as claimed in claim 31, characterized in that the tumor cell expresses at least one cytokine and/or chemokine preferably selected from the group consisting of GM-CSF, G-CSF, IL1, IL2, IL3, IL4, IL5, IL6, IL7, IL8, IL9, IL10, IL11, IL12, IL13, IL14, IL15, IL16, IL17, IL18, IL19, IL20, IL21, IL22, IFNα, IFNβ, IFNγ, Flt3 L, Flt3, TNFα, RANTES, MIP1α, MIP1β, MIP1γ, MIP1δ, MIP2, MIP2α, MIP2β, MIP3α, MIP3β, MIP4, MIP5, MCP1, MCP1β, MCP2, MCP3, MCP4, MCP5, MCP6, 6cykine, Dcck1 and DCDF, particularly preferably selected from the group consisting of GM-CSF, RANTES and MIP1α.

40. The method as claimed in claim 31, characterized in that the tumor cell expresses B7.2 and GM-CSF.

41. The method as claimed in claim 31, characterized in that the tumor cell harbors one or more vector(s) which bring(s) about the expression of one or more of a costimulatory polypeptide.

42. The method as claimed in claim 41, characterized in that the vector comprises nucleic acid sequences which encode the costimulatory polypeptide.

43. The method as claimed in claim 41, characterized in that the vector is of nonviral or viral origin, preferably being derived from the group consisting of AAV, HSV, retrovirus, lentivirus, adenovirus and SV40.

44. The method as claimed in claim 41, characterized in that the vector is present episomally or is integrated into the genome of the cell.

45. The method as claimed in claim 41, characterized in that the vector is derived from AAV.

46. The method as claimed in claim 45, characterized in that the AAV vector is integrated, as a concatamer, in the AAV S1 acceptor site.

47. The method as claimed in claim 41, characterized in that the expression is controlled by a promoter selected from the group consisting of a constitutive, inducible and tissue-specific promoter.

48. The method as claimed in claim 31, characterized in that the tumor cell is proliferation-incompetent, for example as a result of being irradiated or chemically inactivated.

49. The method as claimed in claim 31, characterized in that the pharmaceutical does not comprise any adjuvant.

50. The method as claimed in claim 31, characterized in that the pharmaceutical comprises an adjuvant, preferably CpG.

51. The method as claimed in claim 31, characterized in that the pharmaceutical comprises suitable additives and/or binding agents.

52. The method as claimed in claim 31, characterized in that the patient is a mammal, preferably a human being.

53. The method as claimed in claim 31, characterized in that the vaccine brings about an activation of the lytic activity of NK cells.

54. A method for treating or preventing a tumor in a patient, said method comprising administering to a patient a costimulatory polypeptide-expressing tumor cell for increasing the lytic activity of NK cells, wherein the patient is allogenic with respect to the tumor cell.

55. The method as claimed in claim 54, characterized in that the costimulatory polypeptide is defined as in claim 32.

56. The method as claimed in claim 54, characterized in that the patient possesses at least one tumor, or is to be protected from a tumor, which is of the same type as that from which the tumor cell is derived.

57. The method as claimed in claim 54, characterized in that the tumor cell is defined as in claim 34.

58. The method as claimed in claim 54, characterized in that additionally an adjuvant, preferably CpG, is administered.

59. The method as claimed in claim 54, characterized in that the patient is a mammal, preferably a human being.

60. The method as claimed in claim 54, characterized in that the patient and the tumor cell are partially congruent in their HLA type.
